(19) United States
(12) Patent Application Publication
(10) Pub. No.: US 2003/0202922 A1
(43) Pub. Date: Oct. 30, 2003

(54) Wedge air freshener

(76) Inventor: Mike Farmer, Sioux Falls, SD (US)

Correspondence Address: Vern Schooley,, FULWIDER PATTON LEE & UTECHT, Suite 1550, 200 Oceangate, Long Beach, CA, 90802, US

(21) Appl. No.: 10/132,484

(22) Filed: Apr. 25, 2002

(51) Int. Cl. A61L 9/00

(52) U.S. Cl. 422/306; 422/305

(57) ABSTRACT

An air freshener embodying a fragrance impregnated elongated leg and a laterally projecting resilient wedge to wedge between a pair of louvers.

### FIELD OF THE INVENTION

The present invention relates to air fresheners, in particular, to those adapted to be used in automotive vents.

### DESCRIPTION OF THE PRIOR ART

Air fresheners have been proposed for mounting in a register or the like. It has been common practice to design air fresheners with clips for mounting a housing forming a compartment for removable receipt of fragranced inserts. One such device is shown in U.S. Pat. No. 5,422,078 to Colon.

In my U.S. Pat. Nos. 6,126,906 and 6,264,887, I disclosed an air freshener having a U-shape to define legs for mounting on the opposite sides of a louver and including a portion fabricated of a perforated fragrance material for release of fragrance into an airstream flowed thereover. While commercially successful, experience has shown that such devices have limited utility with different vents having wide ranges of spacing for the louvers.

### SUMMARY OF THE INVENTION

The air freshener of the present invention includes an elongated fragrance impregnated leg to be inserted into an opening between louvers of an air vent, and a wedge combining with the leg and cooperating therewith to be wedged between the louvers to hold the leg frictionally in place.

### BRIEF DESCRIPTION OF THE DRAWINGS

FIG. 1 is a perspective view of a scented wedge air freshener by the present invention;

FIG. 2 is a right-hand end view thereof;

FIG. 3 is a side view thereof mounted between a pair of louvers; and

FIG. 4 is a perspective view in a reduced scale, of the air freshener shown in FIG. 3.

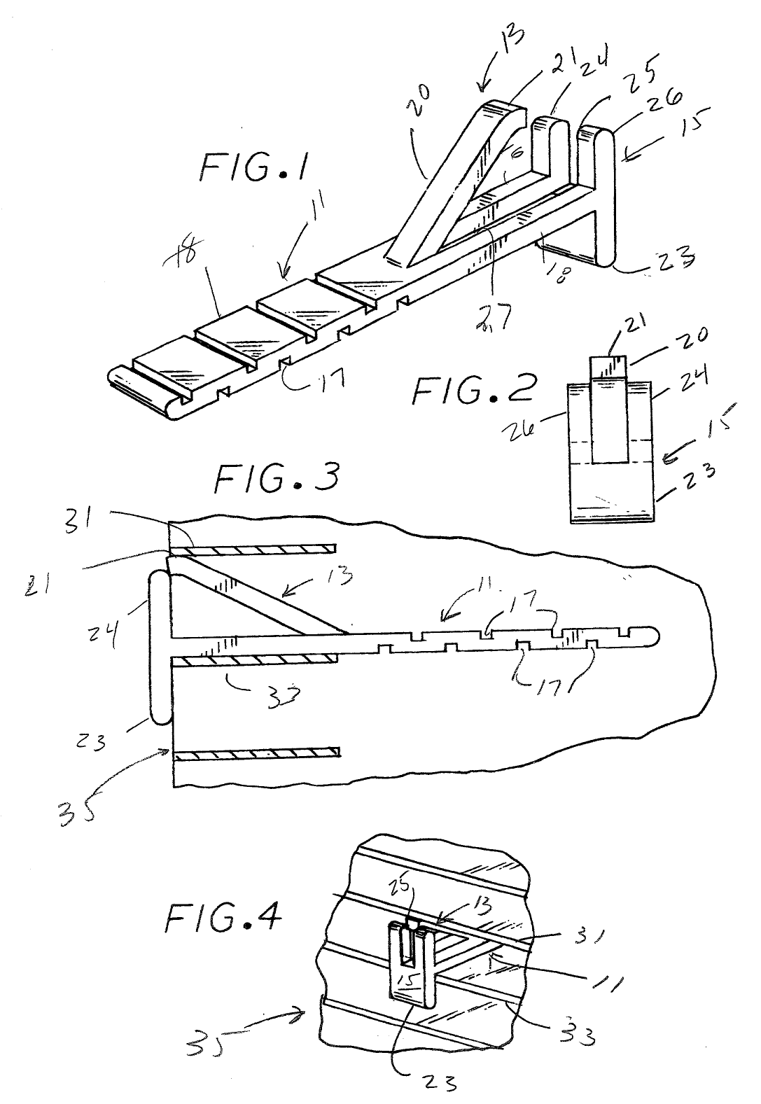

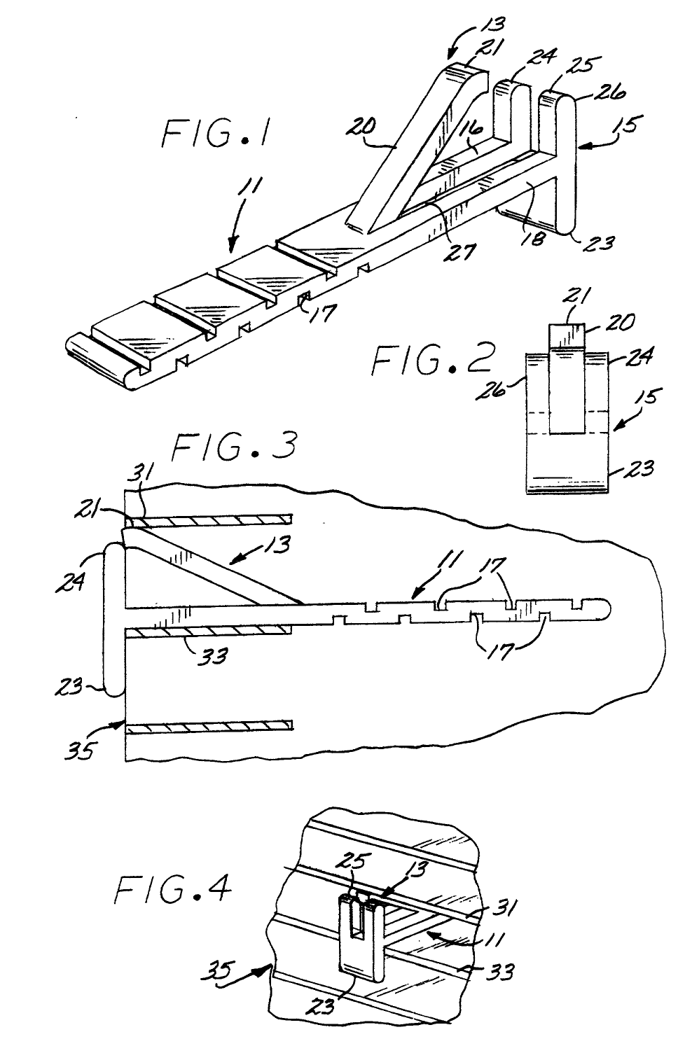

### DETAILED DESCRIPTION OF THE PREFERRED EMBODIMENTS

Referring to FIGS. 1 and 3, the air freshener of the present invention includes, generally, T-shaped body defined by an elongated leg 11 having proximal and distal extremities and mounting on one side thereof a resilient wedge 13 that angles proximally away from such leg. In the preferred embodiment, the air freshener is formed at its proximal end with an enlarged-in-cross section handle, generally designated 15. Also, the leg is preferably formed along its length with oppositely opening, longitudinally spaced apart, transverse fragrant release notches 17.

The air freshener of the present invention may be constructed of any desirable material well-known to those in the art, such as stiff fragrance paper or a polymer such as polyvinyl chloride or polyethlyene. When constructed of a polymer, it is convenient to injection mold my air freshener.

Typically, I use a polymer with a 15 to 25% fragrance load such as that disclosed in my U.S. Pat. No. 6,190,607. I have discovered that to fit the vents of numerous different models of automobiles, the leg 11 may conveniently have a length of about 2½ inches, a width of about ⅜ of an inch, and thickness of about ⅛ of an inch. In the preferred embodiment, the wedge 13 is in the form of an elongated resilient brace 20, projecting proximately and laterally outwardly at angles of about 40° to the longitudinal axis of the leg 11. Such brace is configured at its free approximal extremity with a horizontally projecting bearing surface 21 (FIG. 3). The bearing surface 21, in the unflexed condition of the wedge 13, is preferably spaced about ⅝ of an inch from the side of the leg 11 opposite the wedge 21 to thereby afford a relatively wide bite to accommodate wide spacing between louvers. The relatively small transverse cross-section of such brace then affords sufficient flexibility to allow the free end thereof to flex a quarter of an inch or so laterally inwardly towards the leg to accommodate the varying widths passage between spaced louvers of different automotive vents. The leg 11 is forked at its proximal extremity to form an open ended longitudinal lightening slot 27 about 3/16 of an inch wide between a pair of tines 24 and 26, and underlying the brace 20. In the preferred embodiment of the handle 15 is formed by the head of the T-shape, being about ¾ inch high, ½ wide, bifurcated on the top side to form vertical stems 24 and 26 on opposite sides of an upwardly opening central notch 25 having a width of about 3/16 inch aligned with the slot 27 and disposed under the brace 20 for receipt of the free end of such brace when flexed downwardly to the extreme position shown in FIG. 4. The bottom run of the T-shape of the handle forms a flat plate 23 (FIGS. 1 and 3).

The fragrance release enhancing slots 17 which serve to form indentations in the leg surface may take many different forms, such as corrugations, sawtooth shape, through or blind bores or slots or the like. In the preferred embodiment, slots are about 1/16 wide and are spaced longitudinally about 3/16 apart to form therebetween wide transverse ribs, the slots on the opposite sides of such leg being interposed equidistance between the respective vertical planes of the notches of the opposite sides.

It will be appreciated that the leg 11 may take many different forms and shapes, it only being important that it have a longitudinal dimension and configuration having sufficient surface area, for the polymer or other material selected for construction to release fragrance at a rate sufficient to effectively combat the odor in the automobile for which it is being utilized to freshen the air. In the preferred embodiment the air freshener is generally T-shaped to define the leg and handle.

The wedge 21 may take many different forms, such as for instance the brace 20, a solid triangular wedge, framework with intervening web, rectangular configuration or even being configured with an elliptical outline.

The handle 15 may take numerous different forms, such as the form of a button-head, T-shape, L-shape, or any other desired configuration which will facilitate grasping and which will limit the extent to which the leg 11 may be inserted into the vent.

In operation, the air freshener will typically be injection molded and packaged with, for instance, 2 or 4 to a blister pack. Then, when the user desires to combat the odor, in for instance, his or her automobile, the package may be opened and one of the air fresheners removed. The air freshener may be conveniently inserted between a pair of louvers 31 and 33 of an automotive air vent, generally designated 35 by the user grasping the handle 15 between his or her thumb and forefinger. The air freshener may be inserted by extending the distal end of the leg 11 into the opening between the louvers 31 and 33, sliding it inwardly along the surface of the louver 33 (FIG. 3). As the leg 11 is slid bodily into place, the proximate extremity of the wedge 13, and particularly the bearing surface 21, will engage the other louver 31 of the pair to flex the re-extremity of such wedge downwardly allowing the bearing surface 21 to ride under the bottom surface of the upper louver 31 to thereby exert force driving the leg 11 firmly into engagement with the top surface of the louver 33, thereby serving to maintain the air freshener held frictionally in place in the vent 35. It will be appreciated that for more closely spaced louvers, the free end of the brace 20 may be flexed downwardly to the point where it is received in the upper extent of the notch 25 (FIG. 4) between the stems 24 and 25. The downwardly projecting handle plate 23 will engage the front edge of the lower louver 33 to thus limit the extent to which such leg 11 may be inserted within the vent itself.

When the air conditioner or heater blower is then turned on to flow air out the vent 35, such air will pass over the upper and lower surfaces of the leg 11 and out the space in between the respective louvers. Such air will also flow over and into the respective notches 17, thus passing over the vertical side surfaces of such notches and as well as the respective bottom surfaces of those notches thereby gaining greater exposure to the fragrance oil impregnated into the polymer and enhancing the outgassing of the fragrance and the release rate thereof.

When the vehicle accelerates, it will be appreciated that the inertia of the air freshener will tend to carry such air freshener rearwardly relative to the vent and to the left as viewed in FIG. 3, tending to withdraw it from the vent 35. It will be appreciated, however, that the frictional resistance stemming from the force of the wedge 13 urging the leg 11 against the lower louver 33 will tend to resist that inertial force thereby holding the air freshener in place.

By selecting the size, shape, and resiliency of the wedge 13, the desired wedging force may be selected to cause the air freshener to be held in position during most normal automotive acceleration and turning forces.

In some embodiments, the bottom surface of the leg 11 is textured in the area of the louver 33 and the bearing surface 21 may also be textured to enhance the frictional gripping forces.

From the foregoing it will be apparent that the air freshener of the present invention provides an economical and convenient means for delivering a fragrance at a desired rate to the interior of an automobile.

What is claimed is:

1. A scented wedge air freshener for insertion in an opening of a predetermined width between a pair of louvers of a vent and comprising:
an elongated, fragrance impregnated leg to be inserted in such opening adjacent one of such louvers and formed with proximal and distal ends;
a handle on the proximal end for abutting one of such louvers;
a wedge mounted on such leg and having, in combination with such leg, a width greater than such predetermined width to, upon being inserted in such opening, engage the other such louvers to wedge the leg against the one of such louvers.

2. The scent wedge air freshener set forth in claim 1 wherein:
the wedge is constructed of resilient material.

3. The scent wedge air freshener set forth in claim 1 wherein:
the leg is constructed of polymer.

4. The scent wedge air freshener set forth in claim 1 wherein:
the leg is formed with surface enhancing indentations to increase the surface for emission of the flagrance.

5. The scent wedge air freshener set forth in claim 1 wherein:
the handle projects transversely of the leg to engage one of such levers to limit insertion of such leg

6. The scent wedge air freshener set forth in claim 1 wherein:
the wedge includes a brace mounted centrally and projecting proximally and outwardly therefrom form the leg.

7. The scent wedge air freshener set forth in claim 1 wherein:
the leg and wedge are integral with one another.

8. The scent wedge air freshener set forth in claim 1 wherein:
the leg and wedge are constructed of a polymer.

9. The scent wedge air freshener set forth in claim 1 wherein:
the wedge is constructed of a planer strip having a mounting surface facing laterally in one direction to abut one of such louvers; and
the opposite side of the wedge angles proximally and laterally outwardly in the opposite direction to terminate in a cam surface to abut the other such louver.

10. The scent wedge air freshener set forth in claim 1 wherein:
the leg is formed with ribs defining fragrance release surfaces.

11. The scent wedge air freshener set forth in claim 1 wherein:
the leg is forked on its proximal extremity to form a pair of coextensive tines turned laterally in one direction on their respective proximal ends to form coextensive stems spaced laterally apart to form therebetween a clearance notch; and
the wedge includes an elongated flexible brace mounted centrally on the leg, projecting proximally and laterally outwardly in the one direction to terminate at ifs free in a laterally outwardly facing cam surface, and so configured as to, when flexed laterally inwardly, carry the free end into the clearance slot.

12. The scent wedge air freshener set forth in claim 1 wherein:
the leg is formed on its proximal end with a pair of stems projecting laterally in one direction and spaced laterally apart to form a clearance notch; and
the wedge is in the form of a flexible brace projecting proximally from the medial portion of the leg and laterally outwardly in the one direction to terminate in a free end aligned laterally with the notch and so configured as to be, when the free extremity is flexed laterally inwardly, received in the clearance notch.

13. The scent wedge air freshener set forth in claim 1 wherein:
the leg is formed on its proximal end with laterally projecting hand grasp stems.

14. The scent wedge air freshener set forth in claim 1 wherein:
the leg is formed with longitudinally spaced apart ribs.

15. The scent wedge air freshener set forth in claim 1 wherein:
the leg is formed on its opposite sides with spaced apart ribs.

16. A scent wedge air freshener set forth in claim 1 wherein:
the wedge is flexible and is formed to project proximally from the medial portion of such leg at an angle of substantially 40 degrees to the longitudinal axis of such leg.

17. A scent wedge air freshener set forth in claim 1 wherein:
the wedge and leg are constructed of a polymer.

18. A scent wedge air freshener set forth in claim 1 wherein:
the leg and wedge are constructed of polyethylene.

19. The scent wedge air freshener of claim 1 wherein:
the leg includes surface enhancing means for enhancing the out gassing of the fragrance.

20. A scent wedge air freshener for insertion in an opening of a predetermined width between a pair of louvers of a vent and comprising:
an elongated polymer leg impregnated with a fragrance oil and constructed to be inserted in such opening adjacent one of such louvers and formed with proximal and distal ends;
a polymer wedge mounted on such leg and constructed to, in cooperation with such leg, define a lateral space greater than such predetermined width so that, upon the air freshener being inserted into such opening with the leg riding on one such louver, causes the wedge to engage the other such louver to wedge the leg against the one louver.

21. A scent wedge air freshener for insertion in an opening of a predetermined width between a pair of louvers of a vent and comprising:
a fragrance impregnated elongated body configured with transversely extending surfaces for enhancing out gasses;
such body further including a wedge projecting from the medial portion thereof to angle outwardly and proximally to form a laterally outwardly facing cam surface, such brace being so configured and arranged relative to such body, as to, in its relaxed position space such cam surface a distance greater than such predetermined distance from such distal side of such leg;
such body being further formed proximally with a laterally projecting handle.

22. A scent wedge air freshener for insertion into an automotive air vent having louvers spaced apart a determined distance and comprising:
an elongated fragrance impregnated polymer body to be inserted in such opening adjacent one of such louvers;
such body being formed with a resilient laterally projecting brace to, upon being inserted in such opening engaged on one side with one of such louvers, engage the brace with the other such louver to cooperate in frictionally resisting displacement of such body from such vent when the automobile accelerates;
the body being formed with fragrance release enhancing depressions spaced upon its length.

23. A scent wedge air freshener as set forth in claim 20 for insertion in automotive air vent and wherein:
the wedge is sized such that said lateral space is substantially one inch.
